(19) United States
(12) Patent Application Publication
(10) Pub. No.: US 2006/0241440 A1
(43) Pub. Date: Oct. 26, 2006

(54) Non-thermal acoustic tissue modification

(76) Inventors: Yoram Eshel, Tel Aviv (IL); Ami Glicksman, Petah Tiqwa (IL); Ariel Sverdlick, Tel Aviv (IL); Alexander Falkovich, Ashkelon (IL); Leonid Kushculey, Rehovot (IL); Ilia Vitsnudel, Even Yehuda (IL)

Correspondence Address: DARBY & DARBY P.C., P. O. BOX 5257, NEW YORK, NY, 10150-5257, US

(21) Appl. No.: 11/053,466

(22) Filed: Feb. 7, 2005

(51) Int. Cl. A61B 8/00

(52) U.S. Cl. 600/439

(57) ABSTRACT

A methodology and system for modifying tissue including directing the acoustic beam for a predetermined time duration at a multiplicity of target volumes, which target volumes contain tissue, thereby to modify the tissue in the target volumes while the acoustic beam has a pressure at target volume which lies below a cavitation threshold and the predetermined time duration being shorter than a time duration over which the acoustic beam produces thermal modification of tissue in the target volume

### REFERENCE TO CO-PENDING APPLICATIONS

The subject matter of this application is related to that of copending U.S. patent application Ser. No. 10/021,238 and U.S. Pat. No. 6,607,498 B2.

Computer program listing appendix are submitted herewith on one compact disc and one duplicate compact disc. The total number of compact discs including duplicates is two. The files on the compact discs are software object code for carrying out the preferred embodiment of the invention.

Their names, dates of creation, directory locations, and sizes in bytes of the compact disc are:

TRACKOBJ.HEX of Dec. 28, 2004 located in the directory appendix and of length 2,204,889 bytes.

TRACKDLL.HEX of Dec. 28, 2004 located in the directory appendix and of length 6,236,949 bytes.

The files are referred to herein as Appendix. The material on the compact discs is incorporated by reference herein.

### FIELD OF THE INVENTION

The present invention relates to tissue modification generally and more particularly to non-thermal acoustic tissue modification.

### BACKGROUND OF THE INVENTION

The following U.S. patents and prior art are believed to represent the current state of the art:

U.S. Pat. Nos. 3,637,437; 4,043,946; 4,049,580; 4,110,257; 4,116,804; 4,126,934; 4,169,025; 4,450,056; 4,605,009; 4,826,799; 4,886,491; 4,986,275; 4,938,216; 5,005,579; 5,079,952; 5,080,101; 5,080,102; 5,111,822; 5,143,063; 5,143,073; 5,209,221; 5,219,401; 5,301,660; 5,419,761; 5,431,621; 5,507,790; 5,512,327; 5,526,815; 5,601,526; 5,640,371; 5,884,631; 5,618,275; 5,827,204; 5,938,608; 5,948,011; 5,993,979; 6,039,048; 6,071,239; 6,086,535; 6,113,558; 6,113,559; 6,206,873; 6,309,355; 6,384,516; 6,436,061; 6,573,213; 6,607,498; 6,652,463 B2; 6,685,657 B2; 6,747,180.

PCT International Publication No. WO 2004/014488 A1;

UK Patent No. GB 2 303 552;

Rod J. Rohrich, et al., “Comparative Lipoplasty Analysis of in Vivo-Treated Adipose Tissue”, Plastic and Reconstruction Journal, 105:2152-2158, 2000;

T. G. Muir, et al., “Prediction of Nonlinear Acoustic Effects at Biomedical Frequencies and Intensities”, Ultrasound in Med. & Biol., Vol. 6, pp. 345-357, Pergamon Press Ltd., 1980;

Jahangir Tavakkoli, et al., “A Piezocomposite Shock Wave Generator with Electronic Focusing Capability: Application for Producing Cavitation-Induced Lesions in Rabbit Liver”, Ultrasound in Med. & Biol., Vol. 23, No. 1, pp. 107-115, 1997;

N. I. Vykhodtseva, et al., “Histologic Effects of high Intensity Pulsed Ultrasound Exposure with Subharmonic Emission in rabbit Brain In Vivo”, Ultrasound in Med. & Biol., Vol. 21, No. 7, pp. 969-979, 1995;

Gail R. Ter Haar, et al., “Evidence for Acoustic Cavitation In Vivo: Thresholds for Bubble Formation with 0.75-MHz Continuous Wave and Pulsed Beams”, IEEE Transactions on Ultrasonics, Ferroelectronics, and Frequency Control, Vol. Uffc-33, No. 2, pp. 162-162, March 1986;

D. R. Bacon et al, “Comparison of Two Theoretical Models for Predicting Non-Linear Propagation in Medical Ultrasound Fields”, Phys. Med. Biol. 1989 November; 34(11): 1633-43;

E. L. Carstensen et al, “Demonstration of Nonlinear Acoustical Effects at Biomedical Frequencies and Intensities”, Ultrasound in Med. & Biol., Vol. 6, pp 359-368, 1980.

### SUMMARY OF THE INVENTION

The present invention seeks to provide improved apparatus and methodology for acoustic non-thermal tissue modification.

There is thus provided in accordance with a preferred embodiment of the present invention a method for modifying tissue including the steps of:

providing an acoustic beam; and

directing the acoustic beam at a target volume in a tissue-containing region of a body for a predetermined time duration so as to modify the tissue in the target volume, the acoustic beam having a pressure at the tissue in the target volume which lies below a cavitation threshold thereat, the predetermined time duration being shorter than a time duration over which the acoustic beam produces thermal modification of the tissue in the target volume.

Additionally in accordance with a preferred embodiment of the present invention, there is provided a method for modifying tissue including the steps of:

generating, at a source outside a body, the acoustic beam which generally modifies tissue; and

directing the acoustic beam, from the source outside the body, at a target volume in a tissue-containing region of a body for a predetermined time duration so as to modify the tissue in the target volume, the acoustic beam having a pressure at the tissue in the target volume which lies below a cavitation threshold thereat, the predetermined time duration being shorter than a time duration over which the acoustic beam produces thermal modification of the tissue in the target volume.

Further in accordance with a preferred embodiment of the present invention there is provided a method for modifying tissue including the steps of:

defining a region in a body at least partially by detecting spatial indications on the body; and

directing an acoustic beam at a multiplicity of target volumes within the region, which target volumes contain tissue, the acoustic beam having a pressure at the tissue in the target volume which lies below a cavitation threshold thereat, the predetermined time duration being shorter than a time duration over which the acoustic beam produces thermal modification of the tissue in the target volume, thereby to modify the tissue in the target volumes.

Additionally in accordance with a preferred embodiment of the present invention, there is provided a method for modifying tissue including the steps of:

directing an acoustic beam at a multiplicity of target volumes within the region, which target volumes contain tissue, the acoustic beam having a pressure at the tissue in the target volumes which lies below a cavitation threshold thereat, the predetermined time duration being shorter than a time duration over which the acoustic beam produces thermal modification of the tissue in the target volumes, thereby to modify the tissue in the target volumes; and

computerized tracking of the multiplicity of target volumes notwithstanding movement of the body.

There is additionally provided in accordance with a preferred embodiment of the present invention apparatus for modifying tissue including:

an acoustic beam director, directing an acoustic beam at a target volume in a region of a body containing tissue, the acoustic beam having a pressure at the tissue in the target volume which lies below a cavitation threshold thereat, the predetermined time duration being shorter than a time duration over which the acoustic beam produces thermal modification of the tissue in the target volume; and

a modulator, cooperating with the acoustic beam director to produce the acoustic beam so as to modify the tissue in the target volume.

There is further provided in accordance with a preferred embodiment of the present invention apparatus for modifying tissue including:

a source outside a body generating an acoustic beam, the acoustic beam having a pressure at the tissue in the target volume which lies below a cavitation threshold thereat, the predetermined time duration being shorter than a time duration over which the acoustic beam produces thermal modification of the tissue in the target volume;

an acoustic beam director, which employs the acoustic beam to generally modify tissue in a target volume of a body containing tissue.

There is additionally provided in accordance with a preferred embodiment of the present invention apparatus for modifying tissue including the steps of:

a region definer, defining a region in a body at least partially by detecting spatial indications on the body; and

a director, directing an acoustic beam at a multiplicity of target volumes within the region, which target volumes contain tissue thereby to modify the tissue in the target volumes, the acoustic beam having a pressure at the tissue in the target volumes which lies below a cavitation threshold thereat, the predetermined time duration being shorter than a time duration over which the acoustic beam produces thermal modification of the tissue in the target volumes.

There is still further provided in accordance with a preferred embodiment of the present invention apparatus for modifying tissue including:

a director, directing the acoustic beam at a multiplicity of target volumes within the region, which target volumes contain tissue, thereby to modify the tissue in the target volumes, the acoustic beam having a pressure at the tissue in the target volumes which lies below a cavitation threshold thereat, the predetermined time duration being shorter than a time duration over which the acoustic beam produces thermal modification of the tissue in the target volumes; and

computerized tracking functionality providing computerized tracking of the multiplicity of target volumes notwithstanding movement of the body.

Preferably, directing the acoustic beam generally prevents modification of tissue outside of the target volumes.

In accordance with a preferred embodiment of the present invention, the method also includes acoustic imaging of the region at least partially concurrently with directing the acoustic beam at the target volume.

Preferably, directing includes positioning at least one acoustic transducer relative to the body in order to direct the acoustic beam at the target volume.

The directing may also include varying a focus of at least one acoustic transducer in order to direct the acoustic beam at the target volume. Varying the focus may change the volume of the target volume, and/or the distance of the target volume from the at least one acoustic transducer.

The directing may also include positioning at least one acoustic transducer relative to the body in order to direct the acoustic beam at the target volume.

The method preferably also includes sensing the acoustic beam coupling to an external surface of the body adjacent the target volume.

Preferably, directing takes place from an acoustic transducer located outside of the body.

In accordance with a preferred embodiment of the present invention, the acoustic beam has an initial frequency in a range of 50 KHz-1000 KHz, more preferably in a range of 75 KHz-500 KHz, and most preferably in a range of 100 KHz-300 KHz.

In accordance with a preferred embodiment of the present invention, the acoustic beam has, in the beginning of the treatment area, lost at least dB to harmonic generation.

In accordance with a preferred embodiment of the present invention, the wave form in the treatment area has a “saw tooth” form that creates localized extreme pressure gradients causing the formation of shock waves.

The shock waves modify tissue by creating at least one of the following: apoptosis, necrosis, alteration of chemical and/or physical properties of proteins, alteration of chemical and/or physical properties of lipids, alteration of chemical and/or physical properties of sugars, alteration of chemical and/or physical properties of glycoprotein.

Preferably, the initial modulating provides a duty cycle between 1:2 and 1:250, more preferably between 1:5 and 1:30 and most preferably between 1:10 and 1:20.

In accordance with a preferred embodiment of the present invention, the modulating provides in the treatment area between 1 and 1000 sequential shock waves at an amplitude above a propagating non linear mechanical modification threshold, more preferably between 1 and 100 sequential shock waves at an amplitude above the propagating non linear mechanical threshold and most preferably between 1 and 10 sequential shock waves at an amplitude sufficient for treatment.

Preferably, the modulating includes modulating the amplitude of the acoustic beam over time.

In accordance with a preferred embodiment of the present invention, the total sum of shock waves at a target volume, with an amplitude above a propagating non linear mechanical modification threshold is between 1000 and 100,000, more preferably between 10,000 and 50,000.

In accordance with a preferred embodiment of the present invention, the acoustic beam has an initial shock wave form with a total time of 1 to 10 microsecond.

Preferably, the initial modulating provides a duty cycle between 1:2 and 1:250, more preferably between 1:5 and 1:30 and most preferably between 1:10 and 1:20.

In accordance with a preferred embodiment of the present invention, the modulating provides between 1 and 1000 sequential shock waves at an amplitude above a propagating non linear mechanical modification threshold, more preferably between 1 and 100 sequential shock waves at an amplitude above the propagating non linear mechanical threshold and most preferably between 1 and 10 sequential shock waves at an amplitude above the propagating non linear mechanical threshold.

In accordance with a preferred embodiment of the present invention, the total sum of shock waves at a target volume, with an amplitude above a propagating non linear mechanical modification threshold is between 1000 and 100,000, more preferably between 10,000 and 50,000.

Preferably, directing includes directing the acoustic beam at a multiplicity of target volumes in a time sequence.

In accordance with a preferred embodiment of the present invention, directing includes directing the acoustic beam at plural ones of the multiplicity of target volumes at times which at least partially overlap.

Preferably, at least some of the multiplicity of target volumes at least partially overlap in space.

In accordance with a preferred embodiment of the present invention, the method includes defining the region by marking at least one surface of the body. The method may also include defining the region by selecting at least one depth in the body and/or by detecting tissue in the body and/or by detecting non-modified tissue.

Preferably, directing also includes defining the target volumes as unit volumes of non-modified tissue within the region.

In accordance with a preferred embodiment of the present invention, modulating the acoustic beam so as to modify the tissue in the multiplicity of target volumes proceeds sequentially in time wherein selective modification of tissue in each target volume takes place only following detection of non-modified tissue therein.

Preferably, the method also includes computerized tracking of the multiplicity of target volumes notwithstanding movement of the body.

Preferably, the computerized tracking includes sensing changes in the position of markings on the body and employing sensed changes for tracking the positions of the target volumes in the body.

Preferably, an acoustic conducting layer is located between the acoustic beam director and a contact surface of the body. The acoustic conducting layer typically includes an upper portion located adjacent the acoustic beam director and including a fluid for enhancing cooling during operation of the power source and modulator and a lower portion, located between the upper portion and the contact surface of the body and having an acoustic impedance similar to that of the contact surface.

In accordance with another preferred embodiment there is provided apparatus for modifying tissue including a power source and modulator operative to produce an acoustic beam capable of modifying tissue in a target volume in a tissue-containing region of a body, an acoustic beam director, directing the acoustic beam at the target volume and an acoustic conducting interface located between the acoustic beam director and a contact surface of the body. The acoustic conducting interface includes an upper portion located adjacent the acoustic beam director and a lower portion located between the upper portion and the contact surface of the body. The upper portion includes an acoustic coupling fluid which preferably also enhances cooling during operation of the power source and modulator. The lower portion has an acoustic impedance similar to that of the contact surface. The contact surface of the body is preferably coated with an acoustic coupling medium.

Further in accordance with a preferred embodiment of the present invention, the apparatus for modifying tissue also includes an acoustic coupling medium applicator, supplying an acoustic coupling medium between the acoustic beam director and the body.

Still further in accordance with a preferred embodiment of the present invention, the apparatus for modifying tissue further includes a plurality of sensors operating to determine the extent of acoustic coupling between the acoustic beam director and the body.

Additionally in accordance with a preferred embodiment of the present invention, the apparatus for modifying tissue also includes electronic circuitry associated with the acoustic beam director for storing parameters related thereto.

Preferably, the electronic circuitry stores parameters relating to the operational characteristics of the acoustic beam director.

Further in accordance with a preferred embodiment of the present invention, the apparatus for modifying tissue also includes an interlock circuitry operating to condition operation of the apparatus on receipt of predetermined parameters from the electronic circuitry.

Still further in accordance with a preferred embodiment of the present invention, at least some of the predetermined parameters are stored on an acoustic beam director identification storage medium which when read is supplied to the interlock circuitry for verifying the identity of the acoustic beam director to the interlock circuitry.

There is also provided in accordance with yet another preferred embodiment of the present invention, an apparatus for modifying tissue including a power source and modulator operating to produce an acoustic beam capable of modifying tissue in a target volume in a tissue-containing region of a body, an acoustic beam director, directing the acoustic beam at the target volume and an acoustic coupling medium applicator, supplying an acoustic coupling medium between the acoustic beam director and the body.

There is further provided in accordance with a further preferred embodiment of the present invention, an apparatus for modifying tissue including a power source and modulator operative to produce an acoustic beam capable of modifying tissue in a target volume in a tissue-containing region of a body, an acoustic beam director, directing the acoustic beam at the target volume and a plurality of sensors operative to determine the extent of acoustic coupling between the acoustic beam director and the body.

There is provided in accordance with yet a further preferred embodiment of the present invention, an apparatus for modifying tissue including a power source and modulator operating to produce an acoustic beam capable of modifying tissue in a target volume in a tissue-containing region of a body, an acoustic beam director, directing the acoustic beam at the target volume and electronic circuitry associated with the acoustic beam director for storing parameters related thereto.

Further in accordance with a preferred embodiment of the present invention, the electronic circuitry stores parameters relating to the operational characteristics of the acoustic beam director.

Still further in accordance with a preferred embodiment of the present invention, the apparatus for modifying tissue also includes interlock circuitry operating to condition operation of the apparatus on receipt of predetermined parameters from the electronic circuitry.

Additionally, in accordance with a preferred embodiment of the present invention wherein at least some of the predetermined parameters are stored on an acoustic beam director identification storage medium which when read is supplied to the interlock circuitry for verifying the identity of the acoustic beam director to the interlock circuitry.

### BRIEF DESCRIPTION OF THE DRAWINGS

The present invention will be understood and appreciated more fully from the following detailed description, taken in conjunction with the drawings in which:

FIG. 1 is a simplified pictorial illustration of the general structure and operation of non invasive acoustic non thermal tissue modification apparatus constructed and operative in accordance with a preferred embodiment of the present invention;

FIG. 2 is a simplified block diagram illustration of a preferred pattern of variation of acoustic pressure over time from the acoustic source to the target volume, in accordance with a preferred embodiment of the present invention;

FIGS. 3A and 3B are simplified pictorial illustrations of the appearance of an operator interface display during normal operation and faulty operation respectively;

FIGS. 4A and 4B are respective pictorial and partially cut-away side view illustrations of a patient showing non-uniform distribution of target volumes in a treatment region on a patient;

FIG. 5 is a simplified block diagram illustration of a non invasive acoustic non thermal tissue modification system constructed and operative in accordance with a preferred embodiment of the present invention; and

FIGS. 6A, 6B and 6C are together a simplified flowchart illustrating operator steps in carrying out tissue modification in accordance with a preferred embodiment of the present invention.

Also attached herewith is a CD-ROM appendix which aids in the understanding and appreciation of a preferred embodiment of the invention shown and described herein.

### DETAILED DESCRIPTION OF PREFERRED EMBODIMENTS

A portion of the disclosure of this patent document, which includes a CD-ROM appendix, contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

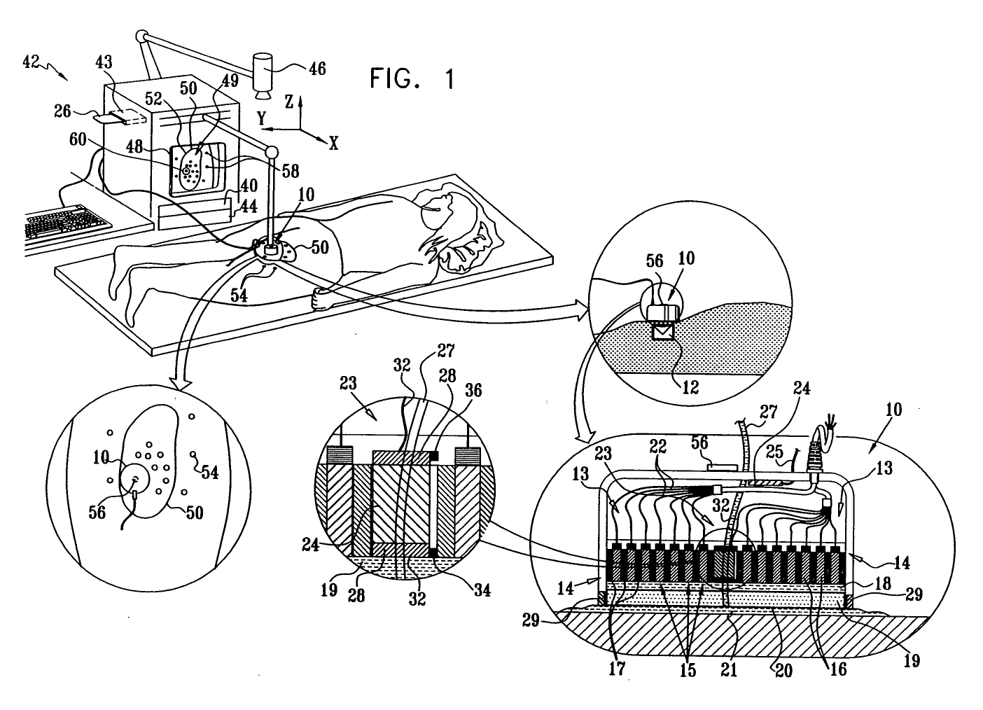

Reference is now made to FIG. 1, which is a simplified pictorial illustration of the general structure and operation of non invasive acoustic non thermal tissue modification apparatus constructed and operative in accordance with a preferred embodiment of the present invention. As seen in FIG. 1, an acoustic beam generator and director, such as an acoustic transducer assembly 10, disposed outside a body, generates the acoustic beam which, by suitable placement of the transducer assembly 10 relative to the body, is directed to a target volume 12 inside the body and is operative to modify tissue therein.

A preferred embodiment of the acoustic beam generator and director useful in the present invention comprises an acoustic therapeutic transducer 13 including a phased array 14 of piezoelectric elements 15 having conductive coatings 16 on opposite surfaces thereof. Individual piezoelectric elements 15 are separated by insulative elements 17. The piezoelectric elements 15 may be of any suitable configuration, shape and distribution.

Typically, an acoustic coupling interface, including first and second layers, is provided between the piezoelectric elements 15 and the body. The first layer, designated by reference numeral 18, preferably is a fluid, such as oil, and preferably serves as both a heat sink and as an acoustic conductor. The second layer, designated by reference numeral 19, preferably is formed of a material, such as polyurethane, which has acoustic impedance similar to that of soft mammalian tissue, and defines a contact surface 20 for engagement with the body, typically via an acoustic coupling medium 21, such as a suitable coupling oil coating the contact surface of the body.

Contact surface 20 may be planar, but need not be. The fluid layer 18 enhances the acoustic contact between piezoelectric elements 15 and polyurethane layer 19. The fluid layer 18 may be circulated during treatment for enhancing cooling.

Suitably modulated AC electrical power is supplied by conductors 22 to conductive coatings 16 to cause the piezoelectric elements 15 to provide a desired acoustic beam output.

In accordance with a preferred embodiment of the present invention, an electronic circuit 24, typically comprising ROM and RAM memories, preferably is mounted in the transducer assembly 10. The electronic circuit 24 preferably is coupled to a control subsystem 42, described hereinbelow, preferably via a connecting cable 25. The ROM preferably stores characteristic parameters of transducer assembly 10, such as its operational frequency its impedance and its maximum stable lifetime. These parameters preferably are also stored on a smart card 26.

The RAM preferably stores operational parameters of transducer assembly 10, such as the number of transmitted acoustic pulses and the cumulative duration of treatments. The information stored in the electronic circuit 24 is employed by interlock circuitry included in subsystem 42 when validating the transducer assembly 10 for operation.

In accordance with a preferred embodiment of the present invention, the acoustic coupling medium 21, such as castor oil, is applied to the contact surface 20 of the transducer 10 and onto the body, typically via a flow tube 27. The flow tube 27 is connected to a suitable acoustic coupling medium storage assembly for supplying the coupling medium 21 to the contact surface 20.

In accordance with a preferred embodiment of the present invention, a plurality pressure sensors 29 are distributed about the circumference of the transducer assembly 10 for sensing engagement between the transducer assembly 10 and the body. Alternatively, pressure sensors 29 may be obviated and the extent of acoustic engagement between the transducer and the body may be determined from an analysis of acoustic signals received by the transducer from the body. In accordance with a preferred embodiment of the present invention an imaging acoustic transducer subassembly 23 is incorporated within transducer 10 and typically comprises a piezoelectric element 24 having conductive surfaces 28 associated with opposite surfaces thereof. Suitably modulated AC electrical power is supplied by conductors 32 to conductive surfaces 28 in order to cause the piezoelectric element 24 to provide an the acoustic beam output. Conductors 32, coupled to surfaces 28, also provide an imaging output from imaging acoustic transducer subassembly 23.

It is appreciated that any suitable commercially available acoustic transducer assembly may be employed or alternatively, imaging acoustic transducer subassembly 23 may be eliminated.

It is further appreciated that various types of acoustic transducers assembly 10 may be employed. For example, such transducers may include multiple piezoelectric elements, multilayered piezoelectric elements and piezoelectric elements of various shapes and sizes arranged in a phase array.

In a preferred embodiment of the present invention shown in FIG. 1, the acoustic beam generator and director are combined in transducer assembly 10. Alternatively, the functions of generating the acoustic beam and directing such beam may be provided by distinct devices.

In accordance with a preferred embodiment of the present invention, a skin temperature sensor 34, such as an infrared sensor, may be mounted alongside imaging acoustic transducer subassembly 23. Further in accordance with a preferred embodiment of the present invention a transducer temperature sensor 36, such as a thermocouple, may also be mounted alongside imaging acoustic transducer subassembly 23.

Acoustic transducer assembly 10 preferably receives suitably modulated electrical power from a power source and modulator assembly 40, forming part of a control subsystem 42. Relevant parameters of the transducer assembly 10 are supplied to interlock circuitry forming part of the control subsystem 42, preferably via smart card 26 which is read by a suitable card reader 43 The interlock circuitry is preferably operative to condition operation of the acoustic transducer assembly 10 on receipt of predetermined parameters from said electronic circuitry. Thus, when an incompatible transducer assembly 10 or a transducer assembly 10 whose stable lifetime has expired is connected, possibly unsafe operation is prevented.

Control subsystem 42 also typically includes a tissue modification control computer 44, having associated therewith a camera 46, such as a video camera, and a display 48. Acoustic transducer assembly 10 is preferably positioned automatically or semi-automatically as by an X-Y-Z positioning assembly 49. Alternatively, acoustic transducer assembly 10 may be positioned at desired positions manually by an operator.

In accordance with a preferred embodiment of the present invention, camera 46 is operative for imaging a portion of the body on which tissue modification is to be performed. A picture of the portion of the patient's body viewed by the camera is preferably displayed in real time on display 48.

An operator may designate the outline of a region 49 containing tissue to be modified. In accordance with one embodiment of the present invention, designation of this region 49 is effected by an operator marking the skin of a patient with an outline 50, which outline 50 is imaged by camera 46 and displayed by display 48 and is also employed by the tissue modification control computer 44 for controlling the application of the acoustic beam to locations within the region. A computer calculated representation of the outline may also be displayed in overlay on display 48, as designated by reference numeral 52. Alternatively, the operator may make virtual markings on the skin, such as by using a digitizer (not shown), which also may provide computer calculated outline representation 52 on display 48.

In addition to the outline representation 52, the functionality of the system of the present invention preferably also employs a plurality of markers 54 which are typically located outside the region 49 containing tissue to be modified, but alternatively may be located inside the region 49 designated by outline 50. Markers 54 are visually sensible markers, which are clearly seen and captured by camera 46 and displayed on display 48. Markers 54 may be natural anatomic markers, such as distinct portions of the body or, alternatively, artificial markers such as colored stickers. These markers are preferably employed to assist the system in dealing with deformation of the region nominally defined by outline 50 due to movement and reorientation of the body during tissue modification. Preferably, the transducer assembly 10 also bears a visible marker 56 which is also captured by camera 46 and displayed on display 48.

Markers 54 and 56 are typically processed by computer 44 and may be displayed on display 48 as respective computed marker representations 58 and 60 on display 48.

The shock waves modify tissue by creating at least one of the following: apoptosis, necrosis, alteration of chemical and/or physical properties of proteins, alteration of chemical and/or physical properties of lipids, alteration of chemical and/or physical properties of sugars, alteration of chemical and/or physical properties of glycoprotein.

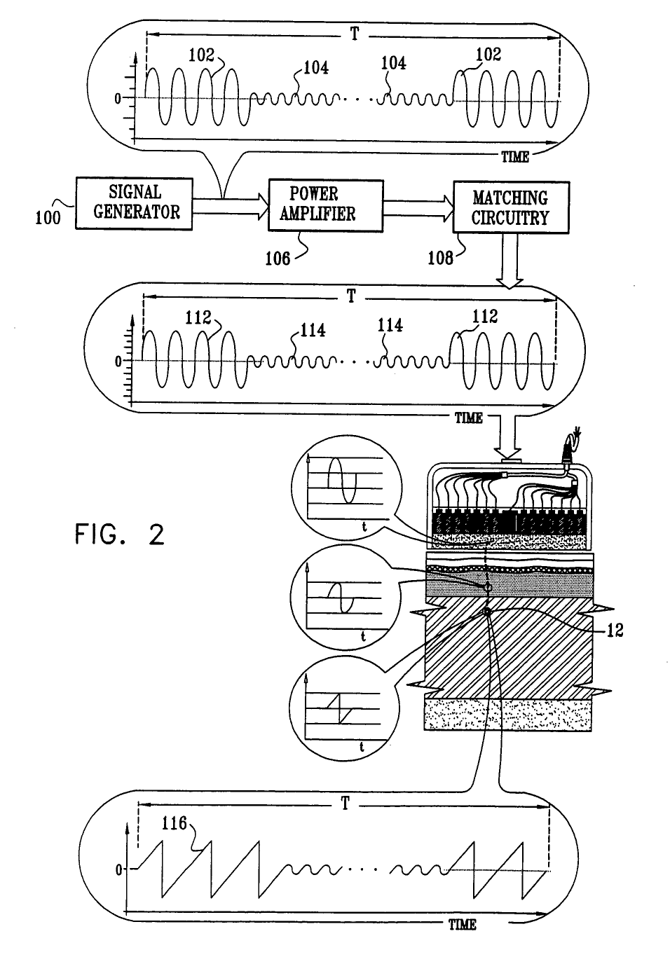

Reference is now made to FIG. 2, which is a simplified block diagram illustration of transducer 10 and portions of preferred power source and modulator assembly 40 (FIG. 1), showing a pattern of variation of acoustic pressure over time at a target volume in accordance with a preferred embodiment of the present invention. As seen in FIG. 2, the power source and modulator assembly 40 preferably comprises a signal generator 100 which provides a time varying signal which is modulated so as to have a series of relatively high amplitude portions 102 separated in time by a series of typically relatively low amplitude portions 104. Each relatively high amplitude portion 102 preferably corresponds to a shock wave in the target volume.

Preferably the relationship between the time durations of portions 102 and portions 104 is such as to provide a duty cycle between 1:2 and 1:250, more preferably between 1:5 and 1:30 and most preferably between 1:10 and 1:20.

Preferably, the maximum of the energy distribution generated as output of signal generator 100 lies in a frequency range from 50 KHz to 1000 KHz, more preferably between 100 KHz and 500 KHz and most preferably between 150 KHz and 300 KHz.

The output of signal generator 100 is preferably provided to a suitable power amplifier 106, which outputs via impedance matching circuitry 108 to an input of acoustic transducer 10 (FIG. 1), which converts the electrical signal received thereby to a corresponding the acoustic beam output. As seen in FIG. 2, the acoustic beam output comprises a time varying signal which is modulated correspondingly to the output of signal generator 100 so as to have a series of relatively high amplitude portions 112, corresponding to portions 102, separated in time by a series of typically relatively low amplitude portions 114, corresponding to portions 104.

Each relatively high amplitude portion 112 has a waveform that is changed during propagation due to nonuniform properties of the medium such that at the target volume 12 (FIG. 1) it has been attenuated by at least 1 dB due to generation of harmonics. The generation of harmonics gives the corresponding waveform at the target volume, indicated by reference numeral 116, a “saw tooth” configuration which produces localized extreme pressure gradients resulting in shock waves.

Relatively low amplitude portions 114 have an amplitude which lies below the treatment threshold and do not produce shock waves at the target volume 12.

In accordance with a preferred embodiment of the present invention, the output of signal generator 100 produces an ultrasonic beam which includes between 1 and 1000 sequential shock waves 102 at an amplitude above a propagating non-linear mechanical modification threshold, more preferably between 1 and 100 sequential shock waves at an amplitude above the propagating non linear mechanical modification threshold and most preferably between 1 and 10 sequential shock waves at an amplitude above the propagating non linear mechanical modification threshold.

In accordance with a preferred embodiment of the present invention, the total number of saw-tooth waveforms applied to a target volume in the course of a treatment is between 1000 and 100,000, more preferably between 10,000 and 50,000. [0

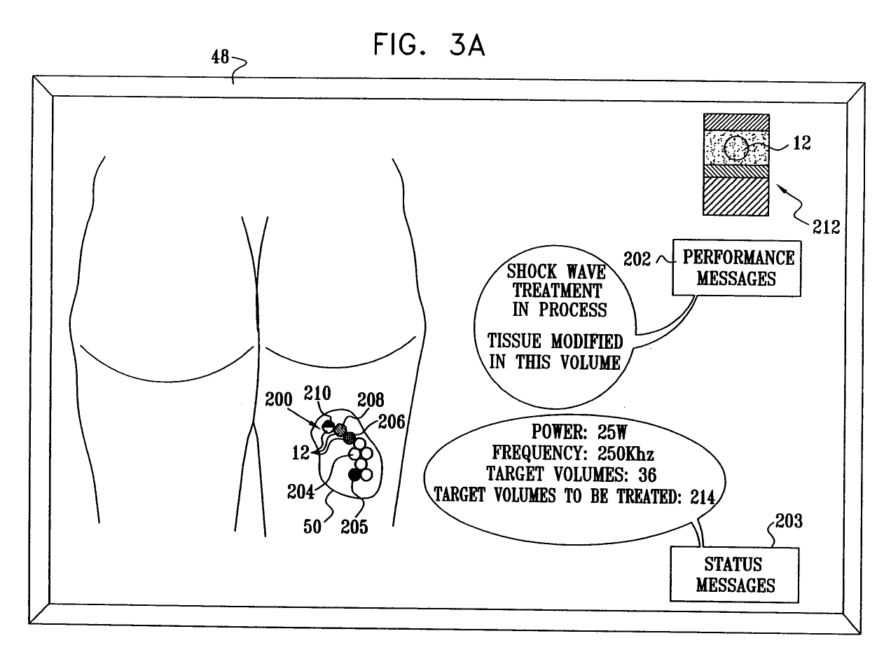

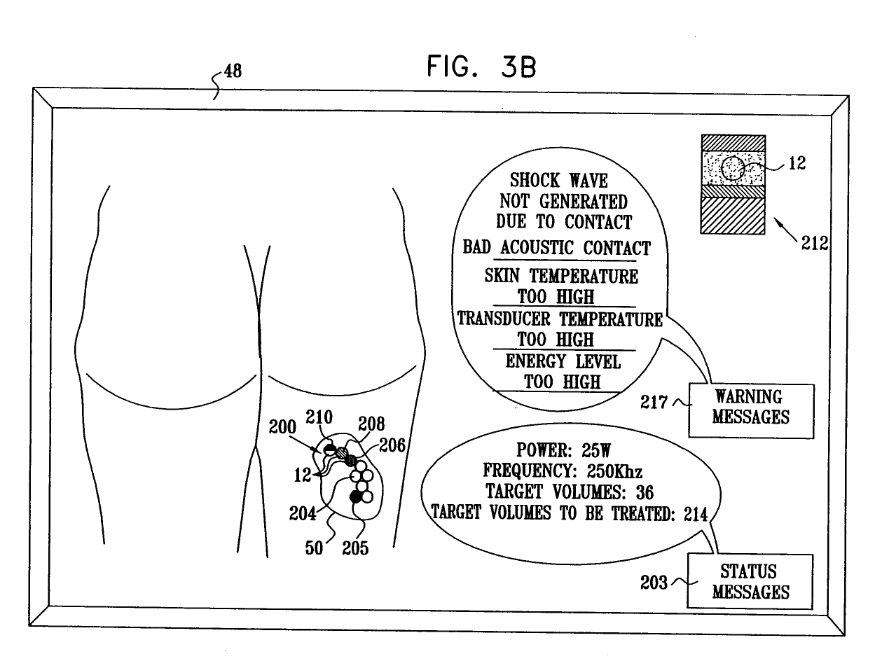

Reference is now made to FIGS. 3A and 3B, which are simplified pictorial illustrations of the appearance of an operator interface display during normal operation and faulty operation respectively. As seen in FIG. 3A, during normal operation, display 48 typically shows a plurality of target volumes 12 (FIG. 1) within a calculated target region 200, typically delimited by outline representation 52 (FIG. 1). Additionally, display 48 preferably provides one or more pre-programmed performance messages 202 and status messages 203.

It is seen that the various target volumes 12 are shown with different shading in order to indicate their treatment status. For example, unshaded target volumes, here designated by reference numerals 204 have already experienced tissue modification. A blackened target volume 12, designated by reference numeral 205 is the target volume next in line for tissue modification. A partially shaded target volume 206 typically represents a target volume, which has been insufficiently treated to achieve complete tissue modification, typically due to an insufficient treatment duration.

Other types of target volumes, such as those not to be treated due to insufficient presence of tissue therein or for other reasons, may be designated by suitable colors or other designations, and are here indicated by reference numerals 208 and 210.

Typical performance messages 202 may include “SHOCK WAVE TREATMENT IN PROCESS” and “TISSUE MODIFIED IN THIS VOLUME”. Typical status messages 203 may include an indication of the power level, the operating frequency, the number of target volumes 12 within the calculated target region 200 and the number of target volumes 12 which remain to undergo tissue modification.

Display 48 also preferably includes a graphical cross sectional indication 212 derived from an acoustic image preferably provided by imaging acoustic transducer subassembly 23 (FIG. 1). Indication 212 preferably indicates various tissues in the body in cross section and shows the target volumes 12 in relation thereto.

Turning to FIG. 3B, it is seen that during abnormal operation, display 48 provides pre-programmed warning messages 214.

Typical warning messages typically may include an indication that shock waves have not been generated due to “BAD ACOUSTIC CONTACT”, “TEMPERATURE TOO HIGH”. The “TEMPERATURE TOO HIGH” message typically relates to the skin tissue, although it may alternatively or additionally relate to other tissue inside or outside of the target volume or in transducer 10 (FIG. 1).

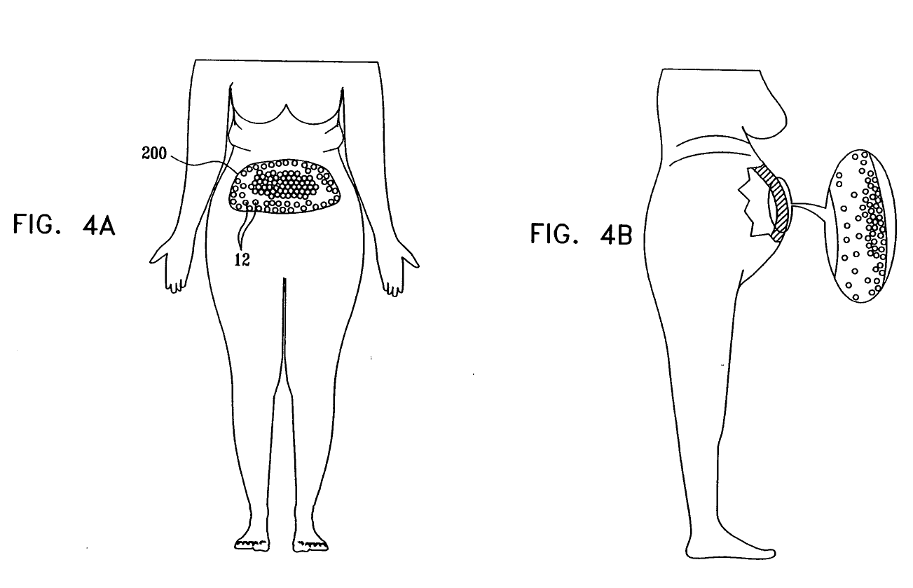

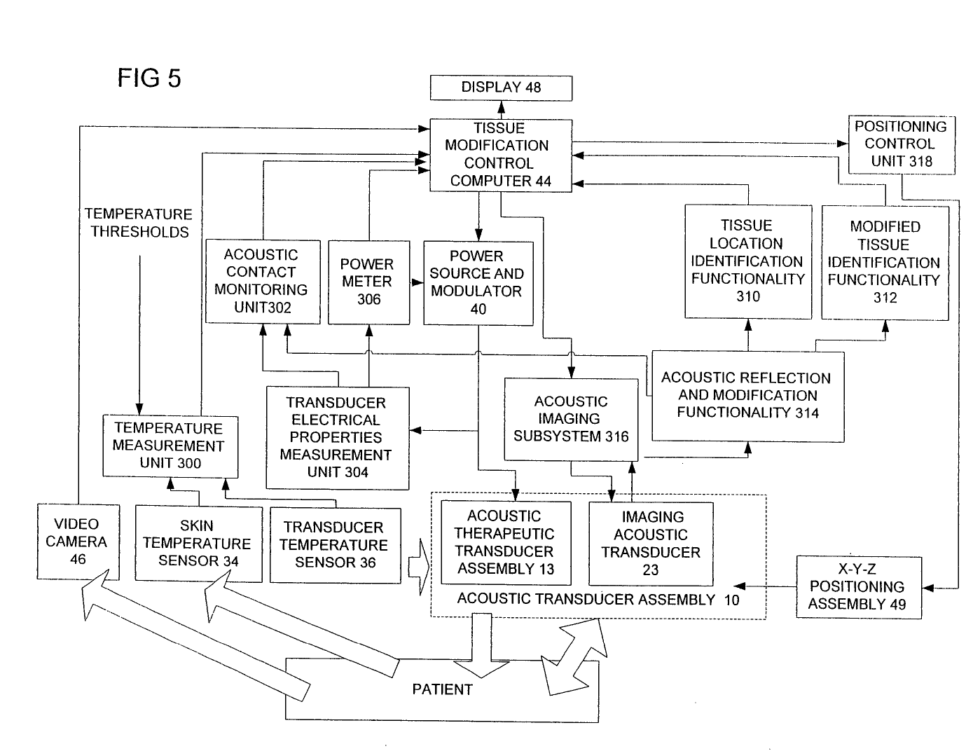

Reference is now made to FIGS. 4A and 4B, which are respective pictorial and partially cut-away side view illustrations of a patient showing non-uniform distribution of target volumes 12 in a treatment region 200 on a patient. It is seen in FIGS. 4A and 4B that the density of target volumes may vary in a target region, both as a function of location relative to a body surface and as a function of depth below a body surface.

Reference is now made to FIG. 5, which illustrates an acoustic tissue modification system constructed and operative in accordance with a preferred embodiment of the present invention. As described hereinabove with reference to FIG. 1 and as seen in FIG. 5, the acoustic tissue modification system comprises a tissue modification control computer 44, which outputs to a display 48. Tissue modification control computer 44 preferably receives inputs from video camera 46 (FIG. 1) and from a temperature measurement unit 300, which receives temperature threshold settings, as well as inputs from skin temperature sensor 34 (FIG. 1) and transducer temperature sensor 36 (FIG. 1). Temperature measurement unit 300 preferably compares the outputs of both sensors 34 and 36 with appropriate threshold settings and provides an indication to tissue modification control computer 44 of exceedance of either threshold. It is a particular feature of the present invention that the temperature threshold settings are selected to be below temperatures which would be required to be attained had a thermal cell destruction functionality been employed, as opposed to the non-thermal tissue modification functionality of the present invention. Typical threshold settings are approximately 38 degrees C. for skin temperature sensor 34 and 40 degrees C. for transducer temperature sensor 36.

An operator directs an acoustic beam towards the target volume 12 in the treatment region 200 by varying the focus of each acoustic beam produced by each piezoelectric element 15 of the phased array 14. Varying the focus of each acoustic beam emitted by the each acoustic element 15, changes the distance of the target volume 12 from each acoustic element 15, as described hereinabove with respect to FIGS. 3A and 3B.

Tissue modification control computer 44 also preferably receives an input from an acoustic contact monitoring unit 302, which in turn preferably receives an input from a transducer electrical properties measurement unit 304. Transducer electrical properties measurement unit 304 preferably monitors the output of power source and modulator assembly 40 (FIG. 1) to acoustic therapeutic transducer assembly 13.

Transducer electrical properties measurement unit 304 preferably compares the output of the power source and modulator 40 with appropriate threshold settings and provides an indication to tissue modification control computer 44 of exceedance of a power level threshold established by the threshold settings. It is a particular feature of the present invention that the power thresholds settings are selected to define a power level threshold which is below a power level characteristic of cavitational cell destruction at a target volume. It is appreciated that the power level characteristic of cavitational cell destruction is substantially higher than the power level employed by the mechanical non-cavitational tissue modification functionality of the present invention.

In accordance with a preferred embodiment of the present invention, the electric power level threshold is significantly less than the power level needed for cavitation in tissue. For example, the power level is 160 Watts for an operating frequency of 250 kHz, when the electric power level threshold found in laboratory experiments for cavitation threshold in water is at least 600 Watts. It is assumed that cavitational cell destruction threshold at the target volume is typically in higher power levels than the threshold for cavitation in water.

Alternatively or additionally, acoustic contact monitoring unit 302 receives an input from acoustic reflection analysis functionality 314.

An output of transducer electrical properties measurement unit 304 is preferably also supplied to a power meter 306, which provides an output to the tissue modification control computer 44 and a feedback output to power source and modulator assembly 40.

Tissue modification control computer 44 also preferably receives inputs from tissue layer identification functionality 310 and modified tissue identification functionality 312, both of which receive inputs from acoustic reflection and modification functionality 314. Acoustic reflection and modification functionality 314 receives acoustic imaging inputs from an acoustic imaging subsystem 316, which operates imaging acoustic transducer subassembly 23 (FIG. 1).

Tissue modification control computer 44 provides outputs to power source and modulator assembly 40, for operating acoustic therapeutic transducer 13, and to acoustic imaging subsystem 316, for operating imaging acoustic transducer subassembly 23. A positioning control unit 318 also receives an output from tissue modification control computer 44 for driving X-Y-Z positioning assembly 49 (FIG. 1) in order to correctly position transducer 10, which includes acoustic therapeutic transducer 13 and imaging acoustic transducer subassembly 23.

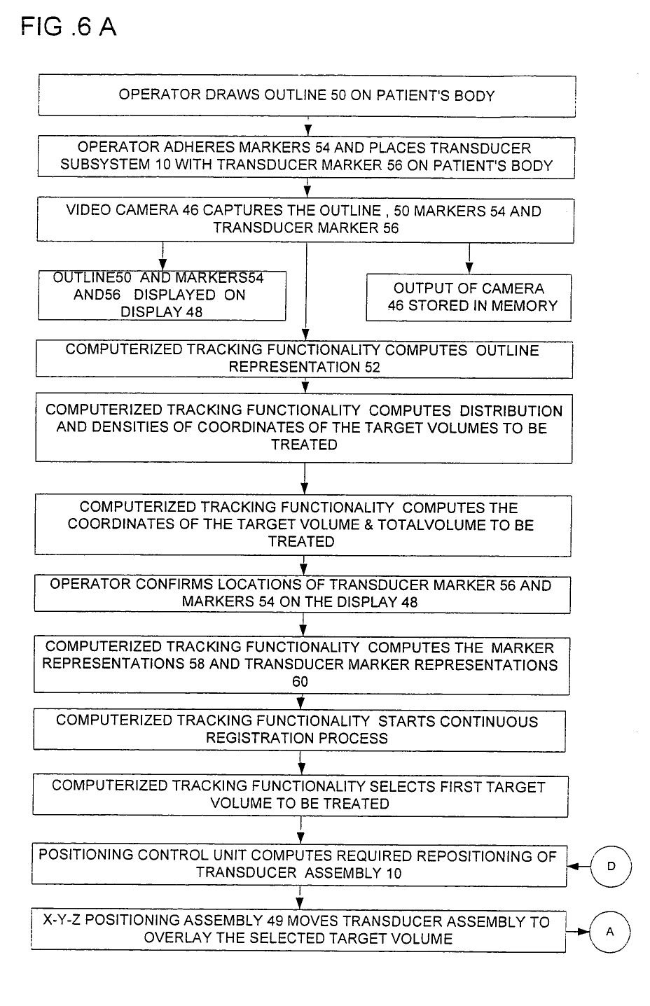

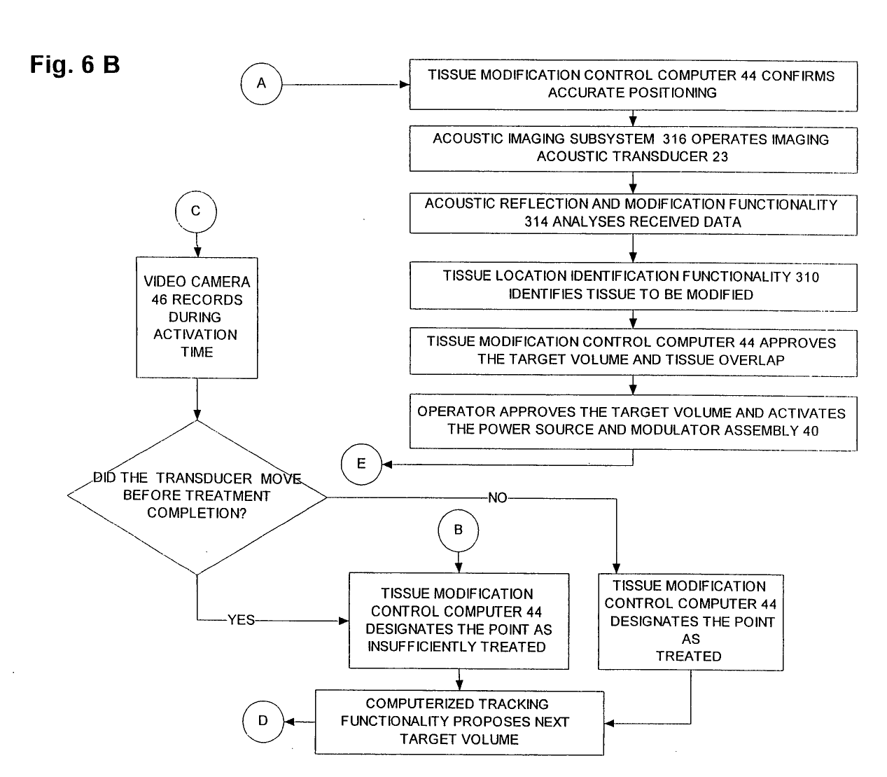

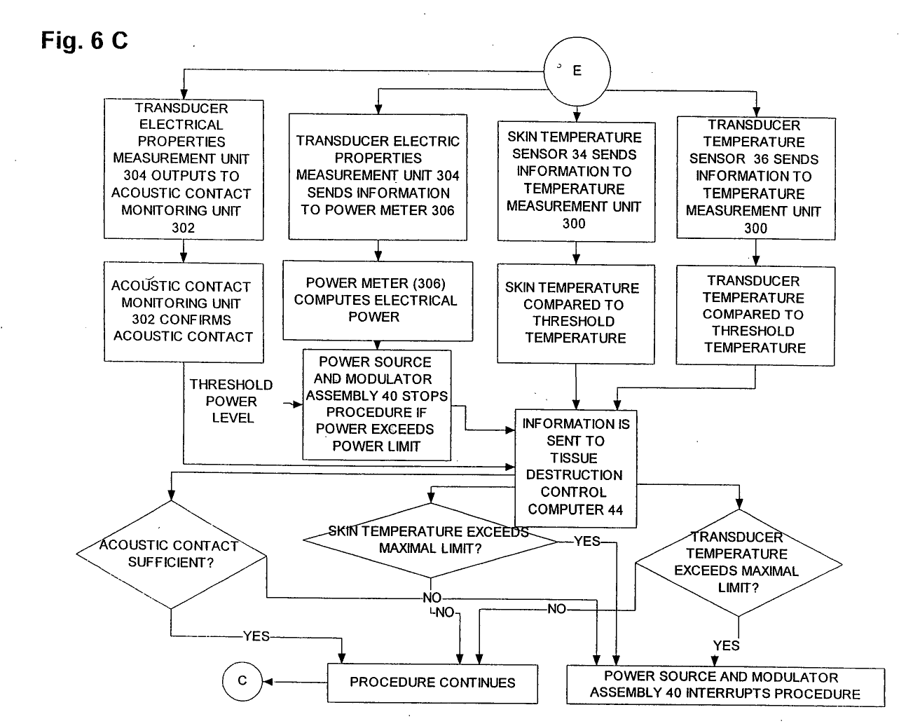

Reference is now made to FIGS. 6A, 6B and 6C, which are together a simplified flowchart illustrating operator steps in carrying out tissue modification in accordance with a preferred embodiment of the present invention. As seen in FIG. 6A, initially an operator preferably draws an outline 50 (FIG. 1) on a patient's body. Preferably, the operator also adheres stereotactic markers 54 (FIG. 1) to the patient's body and places transducer 10, bearing marker 56, at a desired location within outline 50.

Camera 46 (FIG. 1) captures outline 50 and markers 54 and 56. Preferably, outline 50 and markers 54 and 56 are displayed on display 48 in real time. The output of camera 46 is also preferably supplied to a memory associated with tissue modification control computer 44 (FIG. 1).

A computerized tracking functionality preferably embodied in tissue modification control computer 44 preferably employs the output of camera 46 for computing outline representation 52, which may be displayed for the operator on display 48. The computerized tracking functionality also preferably computes the distribution and densities of the target volumes for tissue modification treatment. The distribution of target volumes may be non-uniform both with respect to the body surface and with respect to depth below the body surface, as seen clearly in FIGS. 4A and 4B. The computerized tracking functionality preferably also calculates coordinates of the target volumes and also calculates the total volume to be covered during treatment.

Preferably, the operator confirms the locations of markers 54 and 56 on display 48 and the computerized tracking functionality calculates corresponding marker representations 58 and 60.

In accordance with a preferred embodiment of the present invention the computerized tracking functionality employs markers 54 and marker representations 58 for continuously maintaining registration of outline 50 with respect to outline representation 52, and thus of target volumes 12 with respect to the patient's body, notwithstanding movements of the patient's body during treatment, such as due to breathing or any other movements, such as the patient leaving and returning to the treatment location.

The computerized tracking functionality selects an initial target volume to be treated and positioning control unit 318 (FIG. 5), computes the required repositioning of transducer assembly 10. X-Y-Z positioning assembly 49 repositions transducer assembly 10 to overlie the selected target volume.

Referring additionally to FIG. 6B, it is seen that following repositioning of transducer assembly 10, the tissue modification control computer 44 confirms accurate positioning of transducer assembly 10 with respect to the selected target volume. The acoustic imaging subsystem 316 (FIG. 5) operates imaging acoustic transducer subassembly 23, causing it to provide an output which is supplied by subsystem 316 to acoustic reflection and modification functionality 314.

Acoustic reflection and modification functionality 314 analyses the received data. Based on an output from acoustic reflection and modification functionality 314, tissue location identification functionality 310 identifies tissue to be modified and tissue modification control computer 44 approves the target volume and tissue overlap. Operator may confirm selection of a target volume and activate the power source and modulator assembly 40 (FIG. 1).

Turning additionally to FIG. 6C, it is seen that the following functionalities are provided:

Transducer electrical properties measurement unit 304 provides an output to acoustic contact monitoring unit 302, which determines whether sufficient acoustic contact with the patient is present, preferably by analyzing the current and voltage at therapeutic transducer 13. The output of the monitoring unit 302 is applied to the tissue modification control computer 44.

Transducer electrical properties measurement unit 304 provides an output to power meter 306, which computes the average electrical power received by the therapeutic transducer 13. If the average electrical power received by the therapeutic transducer 13 exceeds a predetermined power level threshold, operation of the power source and modulator assembly 40 may be automatically terminated. As noted above in connection with FIG. 5, the power level threshold is selected in order to avoid cavitation at the target volume. The output of the power source and modulation assembly 40 is applied to the tissue modification control computer 44

Skin temperature sensor 34 measures the current temperature of the skin at transducer subassembly 23 and supplies it to temperature measurement unit 300, which compares the skin temperature to its corresponding threshold temperature. Similarly, transducer temperature sensor 36 measures the current temperature at transducer subassembly 23 and supplies it to temperature measurement unit 300, which compares the transducer subassembly 23 temperature to its corresponding threshold temperature. The outputs of temperature measurement unit 300 are supplied to tissue modification control computer 44.

Should any of the following four conditions occur, the power source and modulator assembly 40 automatically terminates operation of therapeutic transducer 13. Should none of the following conditions occur, the automatic operation of power source and modulator assembly 40 continues:

1. Average electrical power received by the therapeutic transducer 13 exceeds a predetermined threshold;

2. Acoustic contact is insufficient;

3. Skin temperature exceeds threshold temperature; and

4. Transducer 13 temperature exceeds threshold temperature.

Returning to FIG. 6B, it is noted that during automatic operation of power source and modulator assembly 40, video camera 46 preferably records the target region and notes whether the transducer 10 remained stationary during the entire treatment duration of the selected target volume 12. If so, and if none of the aforesaid four conditions took place, tissue modification control computer 44 confirms that the selected target volume was treated. The computerized tracking functionality of tissue modification control computer 44 then proposes a further target volume 12 to be treated.

If, however, the transducer 10 did not remain stationary for a sufficient duration, the selected target volume is designated by tissue modification control computer 44 as having been insufficiently treated.

It is appreciated that by using multiple transducers, a multiplicity of target volumes can be treated sequentially or at least partially overlapping times.

It is also appreciated that the multiplicity of target volumes may at least partially overlap.

The CD-ROM appendix attached herewith is a computer listing of a preferred software implementation of NON-THERMAL ACOUSTIC TISSUE MODIFICATION, constructed and operative in accordance with a preferred embodiment of the present invention.

A preferred method for installing and running the software listing of the CD-ROM appendix is as follows:
1). Provide a PC computer, such as an Intel-based Pentium IV 2.4 GHz computer with Microsoft Windows 2000 Professional operating system, a hard disk with a minimal capacity of 10 GB, 1 available AGP slot, 2 available PCI slots, 1 available USB 2.0 port, 2 available serial ports and a 17″ computer screen.
2). Matrox Orion Frame Grabber Hardware installation/configuration:
   a). Remove/Disable the VGA board present in the PC computer.
   b). Place the Matrox Orion Frame Grabber board available from Matrox Electronic Systems Ltd., 1055 Boul. St-Regis, Dorval, Quebec, Canada H9P 2T4 into an available AGP slot in the PC computer.
   c). Under Microsoft Windows 2000 Professional, on booting the computer, Microsoft Windows' Plug-and-Play system detects a new Multimedia Video Device and requests to assign it a driver. At this point, click Cancel.
   d). Install the Sony FCB-IX45AP Color CCD Camera available from Sony Corp. B&P Systems Co. ISP Dpt. (JAPAN) 4-16-1 Okata, Atsugi-shi, Kanagawa-ken, 243-0021 and connect to the Matrox Orion Frame Grabber.
   e). Set the computer screen impedance switches, red, green, and blue inputs to 75 ohms.
   f). Set the computer screen synchronization inputs to high impedance and external sync mode.
   g). Connect the computer screen to Matrox Orion's 15-pin female VGA output connector (DB-15).
3). Matrox MIL-Lite software (version 7.5) installation:
   a). Run the Matrox MIL-Lite setup.exe program available from Matrox Electronic Systems Ltd. and follow the default prompts.
   b). Run the Matrix Expansion Pack (version 1.0).
   c). Choose “PAL-YC mode of grabbing” when prompted.
4). Advantech PCI-1750 installation:
   a). Place Advantech PCI-1750 I/O card available from Advantech Headquarters No. 1, Alley 20, Lane 26 Rueiguang Road, Neihu District Taipei 114, Taiwan, R. O. C. into an available PCI slot in the PC computer.
   b). Connect UltraShape pulsar's flat cable available from Ultrashape, 30 Habarzel Street, Tel-Aviv 69710 Israel to the Advantech PCI-1750 I/O card.
5). ADVANTECH DA&C Driver Version 2.1b software installation:
   a). Run the Advantech DA&C setup.exe program available from Advantech and follow the default prompts.
   b). Choose “DLL Drivers v1.4c”
   c). Choose “Windows 2000” and complete the installation following default prompts.
   d). Restart the PC computer and run “Device Installation” application
   e). Choose Devise->Setup menu and add “PCI-1750” card
6). MCC PCI DAS4020/12 installation:
   a). Place the MCC PCI DAS4020/12 DAQ card available from Measurement Computing Corporation 16 Commerce Boulevard Middleboro, Mass. 02346 into an available PCI slot in the PC computer.
   b). Connect channels 1,2,3 and trigger to the “U”, “I”, “AE” and “Sync” UltraShape Pulsar's connectors respectively.
7). InstaCal software installation:
   a). Run the InstaCal software setup.exe program available from Measurement Computing Corporation 16 Commerce Boulevard Middleboro, Mass. 02346 and follow the default prompts.
   b). Choose “Install InstaCal” and follow the default prompts.
   c). Restart the PC computer
   d). Run “Instacal” and confirm installation of “PCI DAS4020/12” board
   e). Enter the boards configuration
   f). Choose “A/D Start Trigger” for “Trig/ExtClk BNC Settings->Mode”
   g). Choose “8196 KBytes” for “Contiguous memory settings”
   h). Close the application (clicking “OK” button and choosing “File->Exit” in main menu) and restart the PC computer
8). TiePie Handyscope HS3 installation:
   a). Connect TiePie HandyScope HS3 available from TiePie engineering Koperslagersstraat 37 8601 WL SNEEK The Netherlands into an available USB 2.0 port of the PC computer.
   b). Follow default prompts of “Found new PnP hardware wizard”
   c). Choose “Specify location” and direct the wizard to the “TiePie2K.inf” driver available from the installation CD.
9). Serial communication connections:
   a). Connect UltraShape pulsar's serial connection cable (RS232) available from Ultrashape 30 Habarzel st. Tel-Aviv 69710 Israel to the COM1 of the PC computer.
   b). Connect the Sony CCD camera's serial connection cable (RS232) to the COM2 of the PC computer.
10). Track Software Installation:
   a). Create the following respective directories:
      (1). <Track root>—a root directory for Trackproject
      (2). <Track root>\TrackClinical—contains application configuration and log files.
      (3). <Track root>\TrackClinical\Src—contains source code files
      (4). <Track root>\TrackClinical\Parameters—contains application configuration files
      (5). <Track root>\TrackClinical\Images—contains BMP files for debugging the interior region detection process.
      (6). <Track root>\TrackClinical\Log—contains login related files
      (7). <Track root>\TrackClinical\Timing—contains timing data files for debugging
      (8). <Track root>\TrackClinical\Transducers—contains transducers related files
      (9). <Track root>\TrackClinical\Treatments—contains log files
   b). Copy the file TRACKOBJ.HEX in the root folder stored in the appended CD-ROM into a temporary directory.
   c). Unhex the computer listing TRACKOBJ.HEX using HEX IT V 1.8 or greater by John Augustine, 3129 Earl St., Laureldale, Pa. 19605 creating file TRACKOBJ.ZIP
   d). Decompress the file TRACKOBJ.ZIP using WINZIP version 6.2 or greater, extracting all files into a temporary directory essentially extracting the following object files:
      1) AcquisitionOptions.obj
      2) ColorPickerDlg.obj
      3) ComboBoxTrans.obj
      4) Comm.obj
      5) Common.obj
      6) CompressZip.obj
      7) CustomButton.obj
      8) DIB.obj
      9) DigIO.obj
      10) DigIO_PCI1750.obj
      11) DisplayFuncs.obj
      12) Exception.obj
      13) ExceptionMsgDlg.obj
      14) ImageProc.obj
      15) ImageViwerDlg.obj
      16) InteriorRegion.obj
      17) IOConfigDlg.obj
      18) IOStatusDlg.obj
      19) KeyboardUpFilterInstallUninstall.obj
      20) ListCtrlReport.obj
      21) LoginDialog.obj
      22) Markers.obj
      23) MessageBoxDlg.obj
      24) NewUser.obj
      25) Nodes.obj
      26) Operation.obj
      27) PatientDialog.obj
      28) Picture.obj
      29) SerialComm.obj
      30) Settings.obj
      31) SonyFCBIX.obj
      32) StdAfx.obj
      33) SystemParamsDlg.obj
      34) tp.obj
      35) TP_Acquisition.obj
      36) TrackMain.obj
      37) TrackMainDlg.obj
      38) Transducer.obj
      39) TransducerDlg.obj
      40) TransducerInfo.obj
      41) TransducerTest.obj
      42) TreatLogDlg.obj
      43) TreatmentInfo.obj
      44) TreatmentPreferences.obj
      45) TreatVideoDlg.obj
      46) Utils.obj
      47) VideoMatrox.obj
      48) VideoPositionWnd.obj
      49) ViewDlg.obj
      50) ViewProperties.obj
      51) VinSetup.obj
      52) WinBitmapButton.obj
      53) WinButton.obj
      54) WinRichEditCtrl.obj
   e). Compile the Object code stored in the temporary directory created in step 10d using Microsoft Visual C++.NET compiler version 7.0. The resulting application is created: TRACK.EXE. Move the application file to the <Track root> folder.
   f). Copy the file TRACKDLL.HEX in the root folder stored in the appended CD-ROM into a temporary directory.
   g). Unhex the computer listing TRACKDLL.HEX using HEX IT V1.8 or greater by John Augustine, 3129 Earl St., Laureldale, Pa. 19605 creating the file TRACKDLL.ZIP.
   h). Decompress the file TRACKDLL.ZIP using WINZIP version 6.2 or greater, extracting all files into a temporary directory essentially extracting the following DLL files to the <Track root> folder:
      1) ADSAPI32.DLL
      2) ADSCOMM.DLL
      3) cbw32.dll
      4) FFT.DLL
      5) hs3.dll
      6) hs3f12.hex
      7) Hs3F14.hex
      8) hs3f16.hex
      9) hs3f8.hex
      10) lsprst7.dll
      11) MFC71.dll
      12) msvcp71.dll
      13) msvcr71.dll
      14) PLXAPI.DLL
      15) tmpPrst.dll
   i). To run the Track software, execute the program TRACK.EXE and follow the on-line help to operate the program.

It is appreciated that the software components of the present invention may, if desired, be implemented in ROM (read-only memory) form. The software components may, generally, be implemented in hardware, if desired, using conventional techniques.

It is appreciated that the particular embodiment described in the Appendix is intended only to provide an extremely detailed disclosure of the present invention and is not intended to be limiting.

It is appreciated that various features of the invention which are, for clarity, described in the contexts of separate embodiments may also be provided in combination in a single embodiment. Conversely, various features of the invention which are, for brevity, described in the context of a single embodiment may also be provided separately or in any suitable subcombination.

It will be appreciated by persons skilled in the art that the present invention is not limited by what has been particularly shown and described hereinabove. Rather the scope of the present invention includes both combinations and subcombinations of the various features described hereinabove as well as variations and modifications which would occur to persons skilled in the art upon reading the specification and which are not in the prior art.

1-118. (canceled)

119. A method for modifying tissue comprising the steps of:
providing an acoustic beam; and
directing said acoustic beam at a target volume in a tissue-containing region of a body for a predetermined time duration so as to modify said tissue in said target volume, said acoustic beam having a pressure at said tissue in said target volume which lies below a cavitation threshold thereat, said predetermined time duration being shorter than a time duration over which said acoustic beam produces thermal modification of said tissue in said target volume.

120. A method for modifying tissue according to claim 119 and further comprising the step of providing an acoustic conducting layer located between said acoustic beam director and a contact surface of said body.

121. A method for modifying tissue according to claim 120 and wherein said acoustic conducting layer comprises an upper portion located adjacent said acoustic beam director and comprising a fluid for enhancing cooling during operation of the power source and modulator and a lower portion, located between said upper portion and said contact surface of said body and having an acoustic impedance similar to that of said contact surface.

122. A method for modifying tissue according to claim 119 and wherein said directing the acoustic beam generally prevents modification of tissue outside of said target volume.

123. A method for modifying tissue according to claim 119 and wherein said directing is carried out for a multiplicity of target volumes which are distributed non-uniformly in depth with respect to a surface of said body.

124. A method for modifying tissue according to claim 119 and wherein said directing the acoustic beam generally prevents modification of tissue outside of said target volume.

125. A method for modifying tissue according to claim 119 and also comprising:
acoustic imaging of said region at least partially concurrently with directing said acoustic beam at said target volume.

126. A method for modifying tissue according to claim 119 and wherein said directing comprises positioning at least one acoustic transducer relative to said body in order to direct said acoustic beam at said target volume.

127. A method for modifying tissue according to claim 119 and wherein said directing comprises varying a focus of at least one acoustic transducer in order to direct said acoustic beam at said target volume.

128. A method for modifying tissue according to claim 127 and wherein varying the focus changes the volume of said target volume.

129. A method for modifying tissue according to claim 127 and wherein varying the focus changes the distance of said target volume from said at least one acoustic transducer.

130. A method for modifying tissue according to claim 119 and also comprising sensing the acoustic beam coupling to an external surface of said body adjacent said target volume.

131. A method according to claim 119 and wherein said directing takes place from an acoustic transducer located outside of the body.

132. A method according to claim 119 and wherein the maximum of said energy distribution lies in a frequency range from 50 KHz to 1000 KHz

133. A method according to claim 119 and wherein the maximum of said energy distribution lies in a frequency range from 100 KHz to 500 KHz

134. A method according to claim 119 and wherein the maximum of said energy distribution lies in a frequency range from 150 KHz to 300 KHz.

135. A method according to claim 119 and wherein said acoustic beam has a duty cycle between 1:2 and 1:250.

136. A method according to claim 119 and wherein said acoustic beam has a duty cycle between 1:5 and 1:30.

137. A method according to claim 119 and wherein said acoustic beam has a duty cycle between 1:10 and 1:20.

138. A method according to claim 119 and wherein said acoustic beam has in said target volume between 1 and 1000 sequential shock waves at pressure amplitude above a propagating non linear mechanical modification threshold.

139. A method according to claim 119 and wherein said acoustic beam has in said target volume between 1 and 100 sequential shock waves at pressure amplitude above a propagating non linear mechanical modification threshold.

140. A method according to claim 119 and wherein said acoustic beam has in said target volume between 1 and 10 sequential shock waves at pressure amplitude above a propagating non linear mechanical modification threshold.

141. A method according to claim 119 and wherein accumulated number of shock waves at one said target volume is between 1000 and 100,000.

142. A method according to claim 119 and wherein accumulated number of shock waves at one said target volume is between 10,000 and 50,000.

143. A method according to claim 119 and wherein said acoustic beam has an acoustic signal in the target volume that is decreased by 1 dB in the first harmonic for harmonic generation.

144. Method according to claim 143 and wherein said acoustic signal in the target volume has a “saw-tooth” form.

145. A method according to claim 144 wherein said “saw-tooth” form creates localized extreme pressure gradients causing the formation of shock waves.

146. A method according to claim 119, wherein tissue modification results in cell apoptosis.

147. A method according to claim 119, wherein tissue modification results in cell necrosis

148. A method according to claim 119, wherein tissue modification results in alteration of protein structure.

149. A method according to claim 119, wherein tissue modification results in alteration of protein function.

150. A method according to claim 119, wherein tissue modification results in alteration of sugar structure.

151. A method according to claim 119, wherein tissue modification results in alteration of sugar function.

152. A method according to claim 119, wherein tissue modification results in alteration of lipid structure.

153. A method according to claim 119, wherein tissue modification results in alteration of lipid function.

154. A method according to claim 119, wherein tissue modification results in alteration of glycoprotein structure.

155. A method according to claim 119, wherein tissue modification results in alteration of glycoprotein function.

156. Apparatus for modifying tissue comprising:
a power source and modulator operative to produce an acoustic beam capable of modifying tissue in a target volume in a tissue-containing region of a body; and
an acoustic beam director, directing said acoustic beam at said target volume,
said acoustic beam having a pressure at said tissue in said target volume which lies below a cavitation threshold thereat and impinging on said target volume for a predetermined time duration, said predetermined time duration being shorter than a time duration over which said acoustic beam produces thermal modification of said tissue in said target volume.

157. Apparatus for modifying tissue according to claim 156 and further comprising an acoustic conducting layer located between said acoustic beam director and a contact surface of said body.

158. Apparatus for modifying tissue according to claim 157 and wherein said acoustic conducting layer comprises an upper portion located adjacent said acoustic beam director and comprising a fluid for enhancing cooling during operation of the power source and modulator and a lower portion, located between said upper portion and said contact surface of said body and having an acoustic impedance similar to that of said contact surface.

159. Apparatus for modifying tissue according to claim 156 and wherein said director is operative to direct said acoustic beam at a multiplicity of target volumes which are distributed non-uniformly with respect to a surface of said body.

160. Apparatus for modifying tissue according to claim 156 and wherein said director is operative to direct said acoustic beam at a multiplicity of target volumes which are distributed non-uniformly in depth with respect to a surface of said body.

161. Apparatus for modifying tissue according to claim 156 and wherein said director generally prevents modification of tissue outside of said target volume.

162. Apparatus for modifying tissue according to claim 156 and also comprising:
an acoustic imager providing acoustic imaging of said region at least partially concurrently with directing said the acoustic beam at said target volume.

163. Apparatus for modifying tissue according to claim 156 and wherein said director comprises a positioner, positioning at least one acoustic transducer relative to said body in order to direct said the acoustic beam at said target volume.

164. Apparatus for modifying tissue according to claim 156 and wherein said director varies the focus of at least one acoustic transducer in order to direct said acoustic beam at said target volume.

165. Apparatus for modifying tissue according to claim 164 and wherein varying the focus changes the volume of said target volume.

166. Apparatus for modifying tissue according to claim 164 and wherein varying the focus changes the distance of said target volume from said at least one acoustic transducer.

167. Apparatus for modifying tissue according to claim 156 and wherein said director positions at least one acoustic transducer relative to said body in order to direct said acoustic beam at said target volume.

168. Apparatus for modifying tissue according to claim 156 and wherein said director varies the focus of at least one acoustic transducer in order to direct said acoustic beam at said target volume.

169. Apparatus for modifying tissue according to claim 156 and also comprising a sensor, sensing the acoustic beam coupling to an external surface of said body adjacent said target volume.

170. Apparatus according to claim 156 and wherein said director comprises an acoustic transducer located outside of the body.

171. Apparatus according to claim 156 and wherein the maximum of said energy distribution lies in a frequency range from 50 kHz to 1000 kHz.

172. Apparatus according to claim 156 and wherein the maximum of said energy distribution lies in a frequency range from 100 kHz to 500 kHz.

173. Apparatus according to claim 156 and wherein the maximum of said energy distribution lies in a frequency range from 150 kHz to 300 kHz.

174. Apparatus according to claim 156 and wherein said modulator provides a duty cycle between 1:2 and 1:250.

175. Apparatus according to claim 156 and wherein said modulator provides a duty cycle between 1:5 and 1:30.

176. Apparatus according to claim 156 and wherein said modulator provides a duty cycle between 1:10 and 1:20.

177. Apparatus according to claim 156 and wherein said modulator provides in said target volume between 1 and 1000 sequential shock waves at treatment amplitude.

178. Apparatus according to claim 156 and wherein said modulator provides in said target volume between 1 and 100 sequential shock waves at treatment amplitude.

179. Apparatus according to claim 156 and wherein said modulator provides in said target volume between 1 and 10 sequential shock waves at treatment amplitude.

180. Apparatus according to claim 156 and wherein accumulated number of shock waves at one said target volume is between 1000 and 100,000.

181. Apparatus according to claim 156 and wherein accumulated number of shock waves at one said target volume is between 10,000 and 50,000.

182. Apparatus according to claim 156 and comprising a modulator wherein said modulator modulates the amplitude of said acoustic signal over time.

183. Apparatus according to claim 156 and comprising a modulator modulating the amplitude of said acoustic signal to form in the target volume a decrease by 1 dB in the first harmonic for harmonic generation.

184. Apparatus according to claim 156 and comprising a modulator modulating the amplitude of said acoustic signal to form in the target volume a wave form with a “saw-tooth” form.

185. Apparatus according to claim 184 wherein said “saw-tooth” form creates localized extreme pressure gradients causing the formation of shock waves.

186. Apparatus according to claim 156 wherein tissue modification results in cell apoptosis.

187. Apparatus according to claim 156, wherein tissue modification results in cell necrosis

188. Apparatus according to claim 156, wherein tissue modification results in alteration of protein structure.

189. Apparatus according to claim 156, wherein tissue modification results in alteration of protein function.

190. Apparatus according to claim 156, wherein tissue modification results in alteration of sugar structure.

191. Apparatus according to claim 156, wherein tissue modification results in alteration of sugar function.

192. Apparatus according to claim 156, wherein tissue modification results in alteration of lipid structure.

193. Apparatus according to claim 156, wherein tissue modification results in alteration of lipid function.

194. Apparatus according to claim 156, wherein tissue modification results in alteration of glycoprotein structure.

195. Apparatus according to claim 156, wherein tissue modification results in alteration of glycoprotein function.

196. Apparatus according to claim 156 and comprising a modulator modulating the amplitude of said acoustic signal, taking into account the nonuniformity of the medium to form in the target volume a wave form with a “saw tooth” form that creates thereat localized extreme pressure gradients causing the formation of shock waves.

197. Apparatus for modifying tissue according to claim 156 and comprises also of:
a region definer, defining a region in a body at least partially by detecting spatial indications on said body.

198. Apparatus for modifying tissue according to claim 197 and wherein said definer employs marking at least one surface of said body.

199. Apparatus for modifying tissue according to claim 197 and wherein said definer also employs selection of at least one depth in said body.

200. Apparatus for modifying tissue according to claim 197 and wherein said definer detects tissue in said body.

201. Apparatus for modifying tissue according to claim 197 and wherein said definer defines said region at least partially by detecting non-modified tissue.

202. Apparatus for modifying tissue according to claim 156 and wherein said director also defines said target volumes as unit volumes of non-modified tissue within said region.

203. Apparatus for modifying tissue according to claim 202 and wherein said director proceeds sequentially in time wherein selective modification of tissue in each target volume takes place only following detection of non-modified tissue therein.

204. Apparatus for modifying tissue according to claim 202 and wherein said director also defines said target volumes as unit volumes of tissue within said region.

205. Apparatus for modifying tissue according to claim 202 and wherein said director proceeds sequentially in time wherein selective modification of tissue in each target volume takes place only following detection of tissue therein.

206. Apparatus for modifying tissue according to claim 202 and also comprising computerized tracking functionality providing computerized tracking of said multiplicity of target volumes notwithstanding movement of said body.

207. Apparatus for modifying tissue according to claim 206 and wherein said computerized tracking functionality is operative to sense changes in the position of markings on said body and to employ sensed changes for tracking the positions of said target volumes in said body.

208. Apparatus for modifying tissue according to claim 156 and also comprising an acoustic coupling medium applicator, supplying an acoustic coupling medium between said acoustic beam director and said body.

209. Apparatus for modifying tissue according to claim 156 and also comprising a plurality of sensors operative to determine the extent of acoustic coupling between said acoustic beam director and said body.

210. Apparatus for modifying tissue according to claim 156 and also comprising electronic circuitry associated with said acoustic beam director for storing parameters related thereto.

211. Apparatus for modifying tissue according to claim 210 and wherein said electronic circuitry stores parameters relating to the operational characteristics of said acoustic beam director.

212. Apparatus for modifying tissue according to claim 210 and also comprising interlock circuitry operative to condition operation of the apparatus on receipt of predetermined parameters from said electronic circuitry.

213. Apparatus for modifying tissue according to claim 211 and also comprising interlock circuitry operative to condition operation of the apparatus on receipt of predetermined parameters from said electronic circuitry.

214. Apparatus for modifying tissue according to claim 212 and wherein at least some of said predetermined parameters are stored on an acoustic beam director identification storage medium which when read is supplied to said interlock circuitry for verifying the identity of said acoustic beam director to said interlock circuitry.

215. Apparatus for modifying tissue according to claim 213 and wherein at least some of said predetermined parameters are stored on an acoustic beam director identification storage medium which when read is supplied to said interlock circuitry for verifying the identity of said acoustic beam director to said interlock circuitry.
